(19) United States
(12) Patent Application Publication
(10) Pub. No.: US 2026/0232628 A1
(43) Pub. Date: Aug. 13, 2026

(54) Energy Boosting And Mobility Inducing Formulation For Pets

(71) Applicants: Margaret Jones, Mountain View, HI (US); Marilyn Willis, Florence, AZ (US)

(72) Inventors: Margaret Jones, Mountain View, HI (US); Marilyn Willis, Florence, AZ (US)

(21) Appl. No.: 18/831,873

(22) Filed: Oct. 20, 2025

Related U.S. Application Data
(60) Provisional application No. 63/833,284, filed on Nov. 14, 2024.

(51) Int. Cl. A61K 31/375; A61K 31/05; A61K 31/16; A61K 31/185; A61K 31/194; A61K 31/198; A61K 31/355; A61K 31/385; A61K 31/455; A61K 33/04; A61K 33/06; A61K 33/18; A61K 33/26; A61K 33/30; A61K 33/32; A61K 33/34; A61K 36/18; A61K 36/738; A61K 36/82; A61K 45/06

(52) U.S. Cl. CPC A61K 31/375; A61K 31/05; A61K 31/16; A61K 31/185; A61K 31/194; A61K 31/198; A61K 31/355; A61K 31/385; A61K 31/455; A61K 33/04; A61K 33/06; A61K 33/18; A61K 33/26; A61K 33/30; A61K 33/32; A61K 33/34; A61K 36/18; A61K 36/738; A61K 36/82; A61K 45/06

(57) ABSTRACT

A dry powder formulation, dissolved in water, is designed to be used as a bath for pets, and functions to eliminate stress and anxiety of pets while simultaneously boosting energy and mobility.

### RELATED APPLICATIONS

This application claims priority to U.S. Provisional Application No. 63/833,284 filed on Nov. 14, 2024 entitled “Energy Boosting and Mobility Inducing Formulation For Pets”.

### SUMMARY OF THE INVENTION

In one embodiment, the present invention relates to a formulation comprising Vitamin C, Vitamin E, Vitamin B3, Vitamin B5, alpha-lipoic acid, N-acetylcysteine, green tea extract, trans resveratrol, selenomethionine, Amla fruit extracts, magnesium citrate, magnesium sulfate, rose powder and trace minerals.

In another embodiment, the Vitamin C is in the amount from about 100 to about 400 mg by weight of the formulation. In yet another embodiment, the Vitamin E is in the amount from about 50 to about 200 mg by weight of the formulation. In still another embodiment, the Vitamin B3 is in the amount from about 10 to about 100 mg by weight of the formulation. In still yet another embodiment, the Vitamin B5 is in the amount from about 2 to about 20 mg by weight of the formulation. In a further embodiment, the alpha-lipoic acid is in the amount from about 10 to about 50 mg by weight of the formulation. In another further embodiment, the N-acetylcysteine is in the amount from about 2 to about 20 mg by weight of the formulation. In yet a further embodiment, the green tea extract is in the amount from about 10 to about 50 mg by weight of the formulation. In still a further embodiment, the trans resveratrol is in the amount from about 10 to about 100 mg by weight of the formulation. In still yet a further embodiment, the selenomethionine is in the amount from about 10 to about 100 mcg by weight of the formulation. In another further embodiment, the Amla fruit extract is in the amount from about 10 to about 100 mg by weight of the formulation. In another embodiment, the magnesium citrate is in the amount from about 50 to about 60 g by weight of the formulation. In a further embodiment, the magnesium sulfate is in the amount from about 50 to about 60 g by weight of the formulation. In yet another embodiment, the rose powder is in the amount from about 5 to about 15 mg by weight of the formulation. In still another embodiment, the trace minerals are in the amount from about 20 to about 50 g by weight of the formulation.

In another embodiment, the formulation is dissolved in water before use. In a further embodiment, the formulation dissolved in water is designed to be used as a bath for pets. In yet a further embodiment, the formulation functions to eliminate stress and anxiety of pets while simultaneously boosting energy and mobility. In still a further embodiment, the water is selected from the group consisting of distilled water, tap water, mineral water, spring water, well water, sparkling water, and mixtures and combination thereof.

In another embodiment, the present invention relates to a formulation comprising actives and non-actives. The actives consists of Vitamin C, Vitamin E, Vitamin B3, Vitamin B5, alpha-lipoic acid, N-acetylcysteine, green tea extract, trans resveratrol, selenomethionine, Amla fruit extracts, magnesium citrate, magnesium sulfate, rose powder and trace minerals; and the non-actives consists of cellulose, and lemon balm.

### DETAILED DESCRIPTION OF THE INVENTION

As required, detailed embodiments of the present invention are disclosed herein; however, it is to be understood that the disclosed embodiments are merely exemplary of the invention that may be embodied in various forms. The figures are not necessarily to scale, some features may be exaggerated to show details of particular components. Therefore, specific structural and functional details disclosed herein are not to be interpreted as limiting, but merely as a basis for the claims and as a representative basis for teaching one skilled in the art to variously employ the present invention.

### Vitamin C or Ascorbic Acid:

### Vitamin C Provides the Following Benefits for Pets, Particularly for Canines:

Immune System Support: Vitamin C is a powerful antioxidant that helps boost the immune system by neutralizing cell-damaging free radicals while promoting the production of white blood cells that fight infection. It is also beneficial for sick, stressed, pregnant or aging dogs with depleted Vitamin C.

Joint Health: Vitamin C helps support joint health by producing collagen, a protein vital for connective tissues.

Wound Healing: Vitamin C assists in promoting collagen synthesis and reducing inflammation thereby assisting in healing wounds and repairing body tissues.

Allergy Relief: Vitamin C helps with reducing inflammation and other symptoms associated with allergies by acting as a natural antihistamine.

Skin and Coat Health: The production of collagen helps keep a dog's skin strong and hydrated, resulting in a healthy and lustrous coat.

Stress Reduction: Vitamin C can help lower stress hormones, improving the dog's physiological and behavioral responses to stressful situations.

### Vitamin E:

### Vitamin E Provides the Following Benefits for Pets, Particularly for Canines:

Reduction of inflammation: Vitamin E has been suggested to reduce inflammation of joints, which results from both the common and auto-immune form of arthritis. By reducing inflammation, vitamin E can also be used to reduce pain. For dogs with arthritis, a joint supplement containing vitamin E in conjunction with glucosamine and chondroitin is highly recommended. Vitamin E can also be helpful for vasculitis, an inflammatory process in blood vessels. This often affects the ear tips, eyes and skin, or can affect the whole body secondary to other diseases, such as auto-immune or kidney diseases.

Effect on other diseases: Vitamin E is thought to have many beneficial health effects on other diseases of the liver, skin and eyes and auto-immune diseases such as Lupus and skin disorders. Dogs with diseases of the pancreas, stomach or intestines may have a deficiency in vitamin E and require supplementation. Large breed dogs, particularly Boxers, commonly get a heart disease called Dilated Cardiomyopathy. Many of these dogs have low vitamin E levels and supplementation may be helpful. Eye diseases of the retina, the back portion of the eye which is important in processing vision has been linked to vitamin E deficiency in dogs

Vitamin E is an antioxidant: Antioxidants help prevent damage to cells by eliminating free radicals (molecules that can damage cells, proteins and DNA). Vitamin E may be beneficial for cancers and auto-immune diseases and helps slow down the process of aging.

### Vitamin B3 or Nicotinic Acid:

Vitamin B3 provides the following benefits for pets, particularly for canines:

Energy Metabolism: Vitamin B3 converts carbohydrates, fats and proteins from food into energy.

Skin and Coat: Vitamin B3 helps prevent issues like dermatitis, dryness and hair loss by supporting fatty acid synthesis and functioning as a healthy skin barrier.

Nervous System: Vitamin B3 supports brain function, stress response, and overall nerve function.

Digestive Health: Vitamin B3 aids in the production of stomach acid and bile, which is crucial for proper digestion.

Immune and Hormonal Support: Vitamin B3 regulates certain immune functions and is vital to the production of hormones.

### Vitamin B5 or Pathothenic Acid

### Vitamin B5 Provides the Following Benefits for Pets, Particularly for Canines:

Energy Metabolism: It is a vital component to production of coenzyme A, which is essential for breaking down fats, carbohydrates, and proteins for energy.

Digestive Health: It helps produce digestive enzymes, necessary for digestion and absorption of nutrients.

Skin and Coat Health: It helps attract and retain moisture for the skin and by promoting the synthesis of natural skin fats, it contributes to healthy, hydrated skin and fur.

Nervous System and Brain Function: It plays a key role in creating the neurotransmitters needed for normal brain function. It is also crucial for the production of steroid hormones, including stress-related hormones.

Red Blood Cell Production: Vitamin B5 is necessary for the production of red blood cells, which transports oxygen throughout the body.

### Alpha-Lipoic Acid:

### Alpha-Lipoic Acid (ALA) Provides the Following Benefits for Pets, Particularly for Canines:

Antioxidant and anti-inflammatory: ALA's primary benefit is its strong antioxidant and anti-inflammatory effects, which protect cells from oxidative stress and damage.

Liver support: It supports healthy liver function, as shown by its ability to protect against damage and help with the metabolism of toxins.

Diabetes management: ALA can support normal glucose metabolism and insulin sensitivity, and has been shown to delay the onset of cataracts in diabetic dogs by inhibiting the aldose reductase enzyme.

Cognitive function: Research suggests ALA may help slow the progression of cognitive dysfunction in senior dogs and improve learning.

Joint health: It may help reduce inflammatory markers associated with osteoarthritis.

Nerve protection: ALA is used to help with nerve damage and neuropathy.

### N-acetylcysteine:

### N-Acetylcysteine Provides the Following Benefits for Pets, Particularly for Canines:

### Respiratory Conditions:

Breaks down mucus: It thins thick mucus in the lungs, making it easier for dogs with conditions like bronchitis, collapsing trachea, or asthma to breathe and cough up secretions.

Protects airways: By thinning mucus and improving its clearance, it helps maintain good pulmonary health.

### Antioxidant and Detoxifier:

Boosts glutathione: It is a precursor to glutathione, a powerful antioxidant that helps neutralize free radicals and reduce oxidative stress, which is especially helpful for dogs with chronic diseases or in the first 48 hours of hospitalization for systemic illness.

Detoxifies poisons: It is a crucial antidote for acetaminophen toxicity in dogs, as it helps detoxify the liver from the harmful byproducts of the drug. It can also help with other poisonings, such as carprofen or sulfonamide toxicity.

Supports liver health: It has additional hepatoprotective benefits, including improved oxygen delivery in acute liver failure.

### Antimicrobial Effects:

Some research suggests that NAC may have antimicrobial effects on bacteria commonly found in eye infections

### Green Tea Extract:

### Green Tea Extract Provides the Following Benefits for Pets, Particularly for Canines:

Antioxidant protection: The catechins, particularly EGCG, in green tea help neutralize free radicals, which reduces oxidative stress and may protect against chronic diseases and cell damage.

Anti-cancer properties: Studies suggest green tea extract may inhibit the growth and spread of cancer cells by promoting cell death and preventing new blood vessel growth required by tumors. It can also work synergistically with chemotherapy.

Immune system support: The antioxidants and other beneficial compounds can help boost the immune system and fight off infection.

Skin and coat health: Green tea can be beneficial for skin health by soothing irritation, reducing dandruff, and promoting hair growth.

Oral health: Its antibacterial properties can help reduce bad breath and plaque.

Metabolic support: It may support a healthy metabolism, helping with weight management and lipid balance.

Cardiovascular health: Green tea extract can help support healthy blood pressure and cholesterol levels.

Anti-inflammatory effects: It can help reduce inflammation associated with conditions like arthritis and inflammatory bowel disease.

### Trans-resveratrol:

### Trans-Resveratrol Provides the Following Benefits for Pets, Particularly for Canines:

Antioxidant and anti-inflammatory effects: Resveratrol is a powerful antioxidant that can help fight oxidative stress. It also has anti-inflammatory properties, which can be beneficial for various age-related or chronic conditions.

Cardiovascular support: It may improve blood flow, protect against heart disease by positively affecting platelets and endothelial function, and increase cardiovascular tolerance to stressors like hemorrhage.

Immune system modulation: Studies suggest it can support the immune system by increasing phagocytosis (the ability of immune cells to engulf and clear pathogens) while also regulating other immune responses. It has also been shown to improve stress-related immune markers like IgA.

Stress reduction: Resveratrol may help alleviate chronic stress in dogs by regulating gut microbiota and tryptophan metabolism, leading to reduced anxiety and improved stress-related behaviors.

Potential in cancer therapy: In studies on canine cancer cells, resveratrol has shown promise in inhibiting cancer growth and can potentially enhance the effectiveness of traditional chemotherapy drugs when used as an adjunct therapy.

Other potential benefits: It may improve glucose metabolism and help with metabolic disorders, similar to its effects in humans. Research is also exploring its use in protecting dog sperm during cryopreservation.

### Selenomethionine:

### Selenomethionine Provides the Following Benefits for Pets, Particularly for Canines:

Antioxidant protection: Selenium, in the form of selenomethionine, is a key component of antioxidant enzymes like glutathione peroxidase. This helps to neutralize harmful free radicals, reducing oxidative stress and cellular damage.

Immune system support: By reducing oxidative stress and inflammation, selenomethionine helps support the overall health of the immune system.

Joint health: It is important for cartilage production and can help protect joints from inflammation and the development of arthritis.

Muscle and heart health: Selenium deficiency can lead to muscle weakness and lethargy. Adequate intake is crucial for proper muscle function and can also be linked to heart health.

Reproductive health: Selenium supplementation, often in combination with vitamin E, has been shown to improve semen quality in male dogs, according to the National Institutes of Health.

Thyroid function: The body uses selenium to create enzymes that are critical for converting thyroid hormones (T4) to their active form (T3).

### Amla Fruit Extracts:

### Amla Fruit Extracts Provides the Following Benefits for Pets, Particularly for Canines:

Antioxidants: It can help protect against oxidative stress and support overall health. Its anti-inflammatory properties may help dogs with conditions like arthritis and also support gastrointestinal health.

Immune System: Amla's high Vitamin C content can also boost the immune system and potentially aid in managing allergies.

Healthy Skin and Fur: Antioxidants and Vitamin C in amla can contribute to healthy skin and coat.

Digestion: Antioxidants and Vitamin C in amla support digestion.

### Magnesium Citrate:

### Magnesium Citrate Provides the Following Benefits for Pets, Particularly for Canines:

Digestive health: Relieves constipation by drawing water into the intestines, which softens stool and makes it easier to pass. Promotes bowel movements by helping prompt the muscles in the intestines to move the stool along.

Musculoskeletal and nervous system support: Muscle function: Magnesium is essential for both muscle contraction and relaxation. It helps keep muscles supple and can support muscles during physical exertion. Nerve function: It plays a key role in normal nerve function and helps regulate nerve signals, which can have calming effects.

Bone strength: Magnesium is important for maintaining strong bones and teeth.

Energy production: It helps the body convert food into energy and plays a role in protein synthesis.

### Magnesium Sulfate:

### Magnesium Sulfate Provides the Following Benefits for Pets, Particularly for Canines:

Muscle and joint pain: It can relieve aches, soreness, and stiffness, which is helpful for active dogs or those with arthritis.

Skin issues: It can soothe itchy, irritated skin and may help with minor cuts and abrasions.

Wound healing: It can help cleanse minor wounds and promote healing by reducing inflammation.

### Rose Powder:

### Rose Powder Provides the Following Benefits for Pets, Particularly for Canines:

Joint health: The anti-inflammatory properties of rose powder can help reduce joint pain, stiffness, and inflammation, which is especially beneficial for senior dogs or those prone to joint issues like hip dysplasia.

Immune support: Rose powder is high in antioxidants and vitamin C, which can help boost the immune system and support overall health.

Skin and coat: Essential fatty acids and other nutrients in rose powder help promote healthy skin, improve coat shine, and can reduce dryness, itching, and shedding.

Digestive health: The high fiber content can aid in digestion and help regulate bowel movements.

Recovery and general wellness: It can support recovery after surgery, injury, or exercise and contribute to a dog's overall general health.

### Trace Minerals:

### Trace Minerals Provides the Following Benefits for Pets, Particularly for Canines:

Immune system: Trace minerals such as zinc help boost the immune system, while selenium acts as an antioxidant to protect against cell damage.

Skin and coat: Trace elements such as zinc are vital for a healthy skin barrier and coat quality. Copper helps with iron absorption and melanin production, which affects coat color.

Bone and joint health: Trace elements such as manganese are necessary for bone growth and cartilage formation. Copper is also involved in bone growth. Magnesium supports bone strength and heart function.

Energy and metabolism: Trace elements including iron transports oxygen to organs and muscles and is crucial for energy production. Iodine is essential for producing thyroid hormones that regulate metabolism.

Enzymatic and cellular processes: Many trace minerals are cofactors for enzymes, which are critical for thousands of bodily processes and functions, including cell replication.

One embodiment of the present invention is presented in Table 1 below.

TABLE 1

| | Amount: | |
|---|---|---|
| Actives: | | |
| Vitamin C (as calcium ascorbate) | 100-400 | mg |
| Vitamin E (as d-alpha-tocopheryl acetate) | 50-200 | mg |
| Vitamin B3 (as niacinamide) | 10-100 | mg |
| Vitamin B5 (as Calcium pantothenate) | 2-20 | mg |
| Alpha-lipoic acid | 10-50 | mg |
| N-acetylcysteine (NAC) | 2-20 | mg |
| Green tea extract | 10-50 | mg |
| Trans Resveratrol | 10-100 | mg |
| Selenomethionine | 10-100 | mcg |
| Amla fruit extracts | 10-100 | mg |
| Minerals | 20-30 | g |
| Magnesium citrate | 50-60 | g |
| Magnesium sulfate | 50-60 | g |
| Rose powder | 5-15 | mg |
| Inactive: | | |
| microcrystalline cellulose, and lemon balm. | | |

In another embodiment, the present invention provides a dry powder mix shown in Table 2 designed to be admixed with water prior to use.

TABLE 2

| Ingredients | Range of Amounts | |
|---|---|---|
| Mineral mix | 15-70 | g |
| Vitamin mix | 15-70 | g |
| Magnesium salt | 60-180 | g |

In a further embodiment, the present invention is an antioxidant pet or dog treat as shown in Table 3.

TABLE 3

| | Amount: | |
|---|---|---|
| Dry Ingredients: | | |
| Vitamin C (as calcium ascorbate) | 100-400 | mg |
| Vitamin E (as d-alpha-tocopheryl acetate) | 50-200 | mg |
| Vitamin B3 (as niacinamide) | 10-100 | mg |
| Vitamin B5 (as Calcium pantothenate) | 2-20 | mg |
| Alpha-lipoic acid | 10-50 | mg |
| N-acetylcysteine (NAC) | 2-20 | mg |
| Green tea extract | 10-50 | mg |
| Trans Resveratrol | 10-100 | mg |
| Selenomethionine | 10-100 | mcg |
| Amla fruit extracts | 10-100 | mg |
| Minerals | 20-30 | g |
| Magnesium citrate | 50-60 | g |
| Magnesium sulfate | 50-60 | g |
| Rose powder | 5-15 | mg |

Wet Ingredients:
Pumpkin, peanut butter, wheat flour,
blueberry

In another further embodiment, the wet and dry ingredients in Table 3 are admixed and sprinkled onto any pet or dog treat. Afterwards, it is option to further bake the ingredients in Table 3 onto the pet or dog treat.

Among those benefits and improvements that have been disclosed, other objects and advantages of this invention will become apparent from the following description taken in conjunction with the accompanying drawings. The drawings constitute a part of this specification and include exemplary embodiments of the present invention and illustrate various objects and features thereof.

Numerous modifications and variations of the present invention are possible in light of the above teachings. It is therefore to be understood that within the scope of the attendant claims attached hereto, this invention may be practiced otherwise than as specifically disclosed herein.

What is claimed is:

1. A formulation comprising Vitamin C, Vitamin E, Vitamin B3, Vitamin B5, alpha-lipoic acid, N-acetylcysteine, green tea extract, trans-resveratrol, selenomethionine, Amla fruit extracts, magnesium citrate, magnesium sulfate, rose powder and trace minerals.

2. The formulation of claim 1 wherein said Vitamin C is in the amount from about 100 to about 400 mg by weight of said formulation.

3. The formulation of claim 1 wherein said Vitamin E is in the amount from about 50 to about 200 mg by weight of said formulation.

4. The formulation of claim 1 wherein said Vitamin B3 is in the amount from about 10 to about 100 mg by weight of said formulation.

5. The formulation of claim 1 wherein said Vitamin B5 is in the amount from about 2 to about 20 mg by weight of said formulation.

6. The formulation of claim 1 wherein said alpha-lipoic acid is in the amount from about 10 to about 50 mg by weight of said formulation.

7. The formulation of claim 1 wherein said N-acetylcysteine is in the amount from about 2 to about 20 mg by weight of said formulation.

8. The formulation of claim 1 wherein said green tea extract is in the amount from about 10 to about 50 mg by weight of said formulation.

9. The formulation of claim 1 wherein said trans resveratrol is in the amount from about 10 to about 100 mg by weight of said formulation.

10. The formulation of claim 1 wherein said selenomethionine is in the amount from about 10 to about 100 mcg by weight of said formulation.

11. The formulation of claim 1 wherein said Amla fruit extract is in the amount from about 10 to about 100 mg by weight of said formulation.

12. The formulation of claim 1 wherein said magnesium citrate is in the amount from about 50 to about 60 g by weight of said formulation.

13. The formulation of claim 1 wherein said magnesium sulfate is in the amount from about 50 to about 60 g by weight of said formulation.

14. The formulation of claim 1 wherein said rose powder is in the amount from about 5 to about 15 mg by weight of said formulation.

15. The formulation of claim 1 wherein said trace minerals is in the amount from about 20 to about 50 g by weight of said formulation.

16. The formulation of claim 1 wherein said formulation is dissolved in water before use.

17. The formulation of claim 16 wherein said formulation dissolved in water is designed to be used as a bath for pets.

18. The formulation of claim 1 wherein said formulation functions to eliminate stress and anxiety of pets while simultaneously boosting energy and mobility.

19. The formulation of claim 10 wherein said water is selected from the group consisting of distilled water, tap water, mineral water, spring water, well water, sparkling water, and mixtures and combination thereof.

20. A formulation comprising actives and non-actives, said actives consists of Vitamin C, Vitamin E, Vitamin B3, Vitamin B5, alpha-lipoic acid, N-acetylcysteine, green tea extract, trans resveratrol, selenomethionine, Amla fruit extracts, magnesium citrate, magnesium sulfate, rose powder and trace minerals; and
said non-actives comprising of cellulose, and lemon balm.
